(19) United States
(12) Patent Application Publication
(10) Pub. No.: US 2024/0274234 A1
(43) Pub. Date: Aug. 15, 2024

(54) REMOTE DETECTION OF PATHOGENS

(71) Applicant: Atossa Therapeutics, Inc., Seattle, WA (US)

(72) Inventor: Steven C. QUAY, Seattle, WA (US)

(21) Appl. No.: 18/568,768

(22) PCT Filed: Jun. 24, 2022
(86) PCT No.: PCT/US2022/034934

§ 371 Date: Dec. 8, 2023

Related U.S. Application Data
(60) Provisional application No. 63/215,368, filed on Jun. 25, 2021.

(51) Int. Cl. G16B 30/10; G16B 35/20; G16B 50/40

(52) U.S. Cl. CPC G16B 30/10; G16B 35/20; G16B 50/40

(57) ABSTRACT

Systems and methods for pathogenic genetic sequence identification are provided. A sample of genetic material may be sequenced by a sequencing system. The genetic sequence may be filtered based on sequences designated as not being of interest. Such filtering may yield a filtered sequence subset, which may be compared to sequences that are designated as being of interest. One or more portions of the filtered sequence may be identified as matching the sequences of interest within a predetermined threshold for the sample. Data regarding the matching portions may further be compressed and securely stored in memory. Access to the securely stored data may be limited to authorized persons as defined by applicable rules and policies.

### CROSS-REFERENCE

The present application claims the benefit of U.S. Provisional Application No. 63/215,368, entitled “REMOTE DETECTION OF PATHOGENS,” filed on Jun. 25, 2021, which application is herein incorporated by reference in its entirety for all purposes.

### BACKGROUND OF THE INVENTION

Because of the advancement in current technologies, the ability to work on pathogens (including pathogens of pandemic potential) has been greatly enhanced. For example, a recent paper reported that approximately 30 scientists have been able to grow SARS-COV2 using baker's yeast in approximately 1 week and at a cost less than $5000. The paper also predicted that the timeline could be further compressed to less than a week. Available in the public domain, the paper has been downloaded over 115,000 times. Thus, the technology is now widely available to anyone with an Internet connection, including potential bad actors (e.g., bio-terrorists). As has been seen in the investigation of the SARS-COV2 pandemic, the inability of the World Health Organizing (WHO) investigation team to get any genomic sequencing data during the investigation of the Wuhan Institute of Virology greatly hampered their ability to complete a proper investigation. There is therefore an urgent need to provide improved tools for tracking and investigating potentially dangerous biological research on an ongoing basis, particularly as such research pertains to pathogens.

Pathogens may be identified based on their respective genomes. Genomes associated with pathogens can range in size from about 1 kilobases (kb) to about 300 kb for viruses to more than 14 megabases (Mb) for bacteria, and sequencing may generally be performed in segments. On average, only about 6-10% of the viral genome is non-coding (in contrast to about 50% non-coding for eukaryotic genomes). Genes within the viral genome encode viral capsid proteins (e.g., the envelope proteins and spike proteins of a coronavirus) and viral replication machinery. Sequence conservation between viral proteins varies greatly. For example, the percent sequence identity between a SARS-COV-2 (virus that causes COVID-19) protein and the corresponding protein in SARS-COV (virus that causes SARS) varies from 6% to 96%.

Many viruses, including SARS-COV-2, are ribonucleic acid (RNA) viruses that use RNA rather than deoxyribonucleic acid (DNA) as genetic material. To sequence RNA, the RNA may be reverse-transcribed into DNA and then sequenced rather than sequencing the RNA directly.

### SUMMARY

In various aspects, the present disclosure provides a method for pathogenic sequence identification, the method comprising: receiving a genetic sequence file from a sequencing system, the genetic sequence file including a genetic sequence associated with a sample; filtering the genetic sequence associated with the sample to exclude at least one sequence portion determined not to be of interest, wherein filtering the genetic sequence results in a filtered sequence subset; identifying that at least one portion of the filtered sequence subset includes one or more matches to sequences of interest; and storing data regarding the identified matches in memory.

In some aspects, filtering the genetic sequence includes comparing the genetic sequence to a library of sequences designated as not being of interest, and wherein the at least one excluded sequence portion matches one of the sequences in the library. In some aspects, identifying the matches includes comparing the filtered sequence subset to a library of sequences designated as being of interest, and wherein the identified matches correspond to the sequences in the library.

In some aspects, the method further comprises encrypting the data regarding the identified matches, wherein the stored data is encrypted. In some aspects, the method further comprises retrieving the stored data in response to a request by a requestor, and wherein retrieving the stored data includes decrypting the encrypted data. In some aspects, the method further comprises retrieving the stored data includes verifying that a requestor is authorized to access the stored data. In some aspects, the method further comprises identifying that the matches are associated with a known pathogen.

In some aspects, storing the data regarding the identified matches includes storing an identifier of the known pathogen. In some aspects, storing the data regarding the identified matches includes storing an identifier of one or more sequences of the known pathogen that correspond to the matches. In some aspects, storing the data regarding the identified matches includes storing an identifier of one or more sequences of the known pathogen that do not correspond to the matches.

In various aspects, the present disclosure provides a system for pathogenic sequence identification, the system comprising: a processor; and a memory having programming instructions stored thereon, which, when executed by the processor causes the system to perform operations comprising: receiving a genetic sequence file from a sequencing system, the genetic sequence file including a genetic sequence associated with a sample, filtering the genetic sequence associated with the sample to exclude at least one sequence portion determined not to be of interest, wherein filtering the genetic sequence results in a filtered sequence subset, and identifying that at least one portion of the filtered sequence subset includes one or more matches to sequences of interest.

In some aspects, the processor filters the genetic sequence by comparing the genetic sequence to a library of sequences designated as not being of interest, and wherein the at least one excluded sequence portion matches one of the sequences in the library. In some aspects, the processor identifies the matches by comparing the filtered sequence subset to a library of sequences designated as being of interest, and wherein the identified matches correspond to the sequences in the library. In some aspects, the processor executes further instructions to encrypt the data regarding the identified matches, wherein the stored data is encrypted. In some aspects, the processor executes further instructions to retrieve the stored data in response to a request by a requestor and to decrypt the encrypted data. In some aspects, the processor executes further instructions to retrieve the stored data by verifying that a requestor is authorized to access the stored data. In some aspects, the processor executes further instructions to identify that the matches are associated with a known pathogen.

In some aspects, the memory stores the data regarding the identified matches by storing an identifier of the known pathogen. In some aspects, the memory stores the data regarding the identified matches by storing an identifier of one or more sequences of the known pathogen that correspond to the matches. In some aspects, the memory stores the data regarding the identified matches by storing an identifier of one or more sequences of the known pathogen that do not correspond to the matches.

In various aspects, the present disclosure provides a non-transitory, computer-readable storage medium, having embodied thereon a program executable by a processor to perform a method for pathogenic sequence identification, the method comprising: receiving a genetic sequence file from a sequencing system, the genetic sequence file including a genetic sequence associated with a sample; filtering the genetic sequence associated with the sample to exclude at least one sequence portion determined not to be of interest, wherein filtering the genetic sequence results in a filtered sequence subset; identifying that at least one portion of the filtered sequence subset includes one or more matches to sequences of interest; and storing data regarding the identified matches in memory.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary network environment in which systems and methods for pathogenic sequence identification may be implemented.

FIG. 2 illustrates an exemplary flow of data in a method for pathogenic sequence identification.

FIG. 3 illustrates an exemplary method for pathogenic sequence identification.

FIG. 4A is a block diagram illustrating a computing device, according to example embodiments.

FIG. 4B is a block diagram illustrating a computing device, according to example embodiments.

### DETAILED DESCRIPTION

Embodiments of the present invention include systems and methods of secure and automatic collection and evaluation of genetic sequencing data concerning pathogens of pandemic potential. Such collection and evaluation, which may be performed by local systems, remote systems, or a combination of both remote and local systems, may result in filtered data sets that are usable to track the appearance and evolution of pathogenic strains as they are created or otherwise arise. Such data may be kept in a secured fashion (e.g., to protect the intellectual property rights of the scientists) under a variety of digital access protection schemes, such that only authorized parties (e.g., law enforcement or public health agencies, with proper court authorized warrants relating to forensic and criminal investigations) are allowed access.

In exemplary embodiments, a genetic sequencing machine or system may include or otherwise be associated with a communication interface capable of communicating over communication networks (including the Internet). The association with the communication interface may be mandated or otherwise governed by various legal, statutory, regulatory, or practical considerations regarding genetic sequence data. Thus, the genetic sequences (including raw reads or partial sequences) as identified by the sequencing system may be subject to various analyses as discussed in further detail herein. For example, the raw reads may be analyzed to filter or otherwise exclude human genetic sequences (and other sequences designated as not being of interest), thus preventing storage of human genetic and health data. Examples of types of sequences that may be filtered or otherwise excluded from the genetic sequence data may include human genomic or cDNA sequences, other mammalian genomic or cDNA sequences, yeast genomic or cDNA sequences, (C. Elegans genomic or cDNA sequences, zebrafish genomic or cDNA sequences, or other known non-pathogen genomic or cDNA sequences. The remaining, un-excluded data set may be smaller and more computationally manageable than the full genetic sequence data set. The un-excluded genetic data from the raw reads may further be analyzed to identify sequence homology with conserved regions of known pathogens (e.g., sequences encoding viral replication proteins). The match data may be stored in secured fashion in accordance with various data protection techniques (e.g., passwords, encryption).

In exemplary embodiments of the present invention, systems and methods for pathogenic sequence identification may include sequencing a genetic sample using sequencing systems to obtain an associated genetic sequence. The genetic sequence may be filtered against an established baseline to exclude certain sequences (e.g., known non-pathogenic sequences). The result of the filtering may include one or more subsets of the genetic sequence associated with the sample. For example, a resulting subset may contain sequences that lack sequence identity to known non-pathogenic sequences. The subsets may then be compared to one or more sequences of interest, which may result in one or more matching sequences. Performing the comparison on a subset rather than a full, unfiltered dataset may decrease computation times and reduce the necessary data storage space. The matching sequences may be processed and securely stored in memory, which may include both local storage as well as remote or distributed storage systems (e.g., cloud storage). The matching sequences may further be compressed and encrypted, as well as subject to access controls. Thus, retrieving the matching sequences may require not only system access permissions, but associated keys in order to decrypt the securely stored matching sequences.

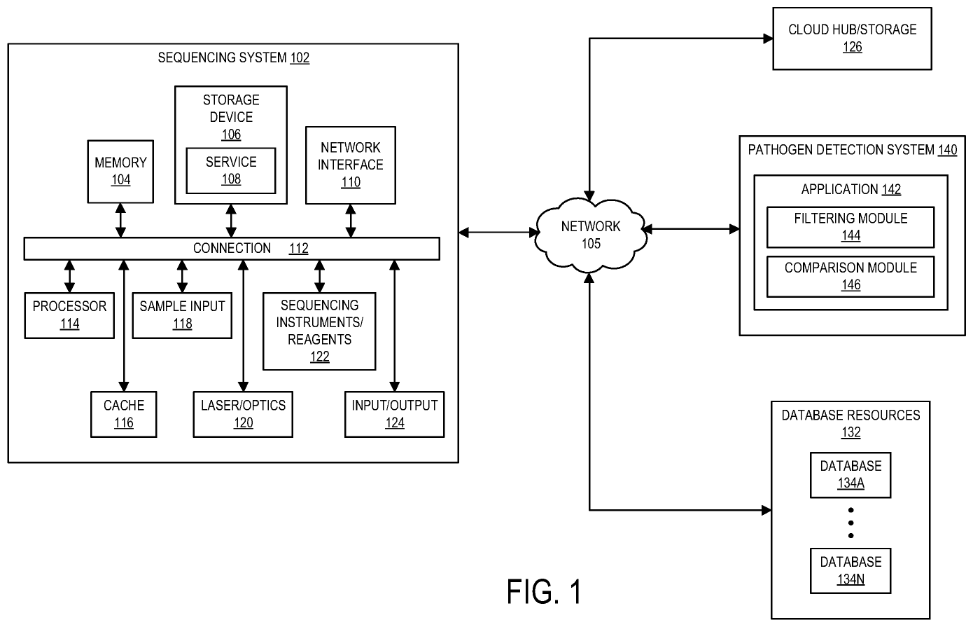

FIG. 1 illustrates an exemplary network environment 100 in which systems and methods for pathogenic sequence identification may be implemented. Network environment 100 may include a sequencing system 102, which may be capable of communicating over communication networks (e.g., network 105). In various embodiments, the sequencing system 102 may communicate with cloud hub/storage systems 126, pathogen detection systems 140 (hosting application 142), and various database resources 132 (e.g., databases 134A-134N).

Sequencing system 102 may be inclusive of sequencing systems known in the art. In an exemplary embodiment, sequencing system 102 may include, integrated with, or in communication with computer processing and storage, which may operate to analyze and store sequence data. As such, the processes described herein may be performed locally by the sequencing system 102 itself, in conjunction with local computing resource, by remote or cloud computing resources, or combinations thereof. Where appropriate, one or more of the computing devices and systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

As illustrated in FIG. 1, sequencing system 102 may include memory 104, storage device 106 (inclusive of storage service 108), network interface 110, connection bus 112, processor 114, cache memory 116, sample input 118, laser/optics 120, sequencing instruments/reagents 122, and output device 124. Depending on the type of genetic sequencing, various components (e.g., laser/optics, sequencing instruments/reagents 122) may be provided in a different configuration or otherwise modified in accordance with the associated parameters of the specific sequencing techniques to be applied.

Sequencing system 102 may be configured to perform genetic sequencing in accordance with methods known in the art. For example, sequencing system 102 may be capable of sequencing by synthesis (e.g., ILLUMINAR sequencing), chain termination sequencing (e.g., Sanger sequencing), single molecule real-time sequencing (e.g., SMRT sequencing), ion semiconductor sequencing (e.g., ION TORRENT™), pyrosequencing (e.g., 454 Life Sciences), combinatorial probe anchor synthesis (e.g., BGISEQ), sequencing by ligation (e.g., SOLID™ sequencing), nanopore sequencing (e.g., Oxford Nanopore), GenapSys sequencing, amplicon sequencing, next generation sequencing, or targeted RNA sequencing.

Sample input 118 may include any device or set of devices for inputting a genetic sample into sequencing system 102 for sequencing. Depending on the specific sequencing methods being used, sample input 118 may include liquid handling robots (robots that can pipet set amounts of different solutions into many different samples simultaneously), gel capillaries, capillary arrays, pipets, tubes, well microplates, cartridges, flow cell chips, or microfluidic pumps.

Various methods for genetic sequencing may use laser/optics 120 to visualize, image, and thereby identify nucleotides (e.g., based on fluorescent or other visual markers) in the genetic sequence of the sample. Laser/optics 120 may include a variety of lasers, optical devices, electric field generators, and sensors (including cameras). Sequencing instruments/reagents 122 may be inclusive of the sequencing instruments and reagents required to perform one or more of the specific sequencing methods discussed herein.

For example, the Sanger sequencing method may use capillary electrophoresis to sequence genetic material. Thus, the sample input 116 for Sanger sequencing/capillary electrophoresis may include gel capillary arrays upon which one or more samples of DNA may be prepared and analyzed. In some embodiments, robots may be used to perform or facilitate subsequent steps, including preparation of the DNA template by amplifying the region of interest (e.g., polymerase chain reaction (PCR)) and ensuring homogenous samples. The prepared DNA template may thereafter be combined with sequencing primers, DNA polymerase, standard nucleotides, and fluorescent terminating nucleotides in a thermal cycler (e.g., PCR machine), which results in elongated DNA sequences terminating in a fluorescent nucleotide.

The fluorescent DNA product may be injected into a gel capillary. In addition, buffers and gel solution packages (e.g., sequencing instruments/reagents 122) may be inserted into sequencing system 102 (like ink/toner cartridges in a printer), which may automatically pipet and mix reagents as needed. The sequencing system 102 may thereafter use laser/optics 120 to perform gel capillary electrophoresis in which a current is applied across the gel capillary (including injected fluorescent DNA product) to cause the differently elongated DNA sequence fragments to move through the gel at a rate that depends on length of fragment (due to negatively charged DNA movement toward positive electrode and the fact that longer fragments move more slowly). Lasers 120 positioned along the capillary may illuminate the fluorescent nucleotides as they pass, and a detector may detect the illuminated fluorescent signals. Time or distance along capillary of signal detection determines position of nucleotide, while fluorescence color may determine identity of the nucleotide.

Software may be used to convert the sensor signals to DNA sequences. The output (provided via input/output 124) may include files with the nucleotide sequence in ATCG letter codes and a 4-color intensity profile (representing the fluorescence signals from each nucleotide) with the corresponding nucleotide letter underneath each intensity peak.

Other embodiments of sequencing system 102 may use next generation sequencing methods, which may include preparation of the DNA template via PCR of a sequence or region of a sequence of interest. Unlike Sanger sequencing, next generation sequencing can accommodate a heterogeneous sample of DNA fragments, as well as longer fragments. Sample input 116 may be combined with sequencing instruments/reagents 122 by way of a flow cell and sequencing cartridge. The flow cell and sequencing cartridge-which may include the DNA template, primers, DNA polymerase, and reagents-may be inserted cartridge into a sequencer (for fully automated machines). Cluster formation may occur, resulting in amplification of DNA on flow cell surface. DNA with adapter sequences may be adhered to a flow cell covered in oligo sequences that bind to the adapter sequences.

The DNA may be amplified, for example using thermal or isothermal amplification, resulting in clusters of amplified DNA bound to the surface (“bridge amplification”) using two types of surface-bound oligo nucleotides. The first may adhere to a first adapter sequence on one end of the DNA, and the second may adhere to the second adapter sequence on the other end of the DNA. The DNA template may therefore form a “bridge” between two surface oligonucleotides. The template may be replicated, resulting in two complementary strands bound to the surface by one end. Each cluster on the chip contains identical sequences. Different clusters may have different sequences, allowing massively parallel sequencing.

Sequencing may thereafter be performed based on the fluorescent nucleotides that are incorporated into elongating chain during DNA synthesis. Unlike Sanger sequencing, the elongating nucleotides may be fluorescent or otherwise labeled in next generation sequencing, not just the terminating bases. Thus, each nucleotide may be fluorescently or otherwise detected (using a laser and a sensor). The number of nucleotide/detection cycles determines the position within the sequence, and the fluorescent wavelength determines the identity of the nucleotide. In some embodiments, index reads may be collected using index primers, which may be used for calibration/control purposes.

The result of next generation sequencing may therefore include millions of “reads” from different fragment clusters on the surface (one fragment per cluster on the flow cell, forward and reverse reads for each fragment). Overlapping ends of fragments may be used to align into a longer sequence (this would be done in the sequencing software, but a user may be able to input/adjust parameters). Finally, the output (provided via input/output 124) may include files with ATCG codes.

With the exception of single molecule real-time sequencing and nanopore sequencing, many of the above methods can only sequence up to a few hundred bases per read (some even less). As a result, sequencing of long sequences (e.g., viral genomes) may be performed in segments. One difference between sequencing methods is how nucleotides are sequentially identified (e.g., using fluorescent nucleotides or detecting a change in electrical current). Other differences include the length of sequence that can be detected, the sensitivity (some methods can sequence single DNA molecules, others require many copies to generate a detectable signal), whether they can directly sequence RNA, etc.

Generally, to sequence a region of a target DNA sequence, a primer (e.g., DNA piece about 18-24 bases long) that binds to the target DNA at a specific point may be selected. A region up to about 500 bases downstream of the primer binding site can be sequenced per sequencing reaction. For sequences longer than about 500 bases, multiple sequencing reactions may be performed using different primers that bind at different sites along the target DNA sequence. The fragments sequenced in the multiple sequencing reactions should overlap to allow for full sequence reconstruction. The full target DNA sequence may be reconstructed by matching the overlapping sequences to determine the fragment order.

In Sanger sequencing, sequencing is performed using terminating fluorescent bases. The terminating bases end DNA elongation when incorporated into a DNA sequence. The target DNA acts as a template for replication initiating from the primer sequence, and elongation is performed using a mixture of nucleotides containing regular A, T, C, and G nucleotides mixed with a small percent of terminating A, T, C, and G nucleotides. Each of the terminating A, T, C, and G nucleotide are labeled with a different colored fluorescent marker. DNA elongation proceeds as normal until a terminating base is incorporated, at which point termination stops. Since there is only a small percent of terminating bases, this results in a population of polynucleotides with a range of sequence lengths, each ending with a fluorescently labeled nucleotide. The sequence is determined by measuring the length of each polynucleotide (using capillary gel electrophoresis), and the color of the corresponding fluorophore (using a laser and a fluorescence detector). The length of the sequence tells you where the nucleotide is positioned, and the color of the fluorophore tells you whether the nucleotide is A, T, C, or G.

Depending on the sequencing method, multiple sequencing repeats, or “reads,” are often performed to get more reliable data. “Raw reads” refers to each sequencing read before they are compiled into a final (more accurate) sequence. Some types of sequencing (such as ILLUMINA R or other next generation sequencing) take a very large number of raw reads to determine the final sequence.

In some embodiments, the memory 104 may include non-transitory computer-readable storage media, which may refer to any medium or media that participate in providing instructions to a processor or central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, read-only memory (ROM), random-access memory (RAM), PROM, EPROM, a FLASHEPROM, and any other memory chip or cartridge, and other such memory technologies known in the art including, but not limited to, those described herein. The memory 104 may be referred to herein as system memory or computer system memory. The memory 104 may include, at various times, elements of an operating system, one or more applications, data associated with the operating system or the one or more applications, or other such data associated with the sequencing system 102. As such, the memory 104 can include multiple different types of memory with different performance characteristics.

In some embodiments, the storage device 106 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof. As described herein, the storage device 106 can include hardware and/or software services such as service 108 that can control or configure the processor 114 to perform one or more functions including, but not limited to, the methods, processes, functions, systems, and services described herein in various embodiments. In some embodiments, the hardware or software services can be implemented as modules. As illustrated, the storage device 106 can be connected to other parts of the sequencing system 102 using the system connection bus 112. In an embodiment, a hardware service or hardware module such as service 108, that performs a function can include a software component stored in a non-transitory computer-readable medium that, in connection with the necessary hardware components, such as the memory 104, storage device 106, connection bus 112, processor 114, cache 116, input/output 124, and so forth, can carry out the functions such as those described herein.

The sequencing system may further include a network interface device such as the network interface 110. The network interface can include one or more of a modem or other such network interfaces including, but not limited to those described herein. Network interface 110 may be integrated into part of the sequencing system 102 or may be provided separately. The network interface 110 can include one or more of an analog modem, Integrated Services Digital Network (ISDN) modem, cable modem, token ring interface, satellite transmission interface, or other interfaces for coupling a computer system to other computer systems. Network interface 110 may be inclusive of interfaces used to communicate using wired or wireless networks (e.g., network 105), including local area networks (LANs) and wide area networks (WANs, inclusive of the Internet). Thus, network interface 110 allows sequencing system 102 to communicate with other computing devices, including remote and online computing systems (e.g., pathogen detection system 140).

Connection bus 112 may be configured to carry data between various system components of sequencing system 102. For example, connection bus 112 allows for exchange of data, as well as processing instructions, between processor 114 and the various storage devices illustrated and described herein, including memory 104, storage device 106 (and service 108), and cache memory. Connection bus 112 may also be used to relay data to and from network interface 110, sample input 118, laser/optics 120, sequencing instruments/reagents 112, and input/output 124.

The processor 114 can include any general-purpose processor and one or more hardware or software services, such as service 108 stored in storage device 106, configured to control the processor 114 as well as a special-purpose processor where software instructions are incorporated into the actual processor design. The processor 114 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 114 can be a microprocessor, a microcontroller, a digital signal processor (“DSP”), or a combination of these and/or other types of processors. In some embodiments, the processor 114 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units. Using modules, methods and services such as those described herein, the processor 114 can be configured to perform various actions such as those associated with methods described herein.

In some embodiments, the sequencing system 102 may include a cache 116 of high-speed memory connected directly with, in close proximity to, or integrated as part of the processor 114. Data may be copied from the memory 104 and/or the storage device 106 to the cache 116 for quick access by the processor 114. In this way, the cache 116 can provide a performance boost that decreases or eliminates processor delays in the processor 114 due to waiting for data. In some embodiments, the cache 116 may include multiple types of cache including, for example, level one (L1) and level two (L2) cache.

Sequencing system 102 may further include one or more input and/or output (I/O) devices 124. The I/O devices 124 can include, by way of example but not limitation, a keyboard, a mouse, a printer, a scanner, a display device, and other such components. Other examples of input devices and output devices are described herein. In some embodiments, input/output devices 124 can be implemented as an integrated part of sequencing system 102 or as a separate (e.g., peripheral) device.

As illustrated in FIG. 1, sequencing system may further be in communication with cloud hub/storage device 126, pathogen detection system 140 (including application 142), and database resources 132 (including various database 134A-134N), each of which may include any type of server or other computing device as is known in the art, including standard hardware computing components such as network and media interfaces, non-transitory computer-readable storage (memory), and processors for executing instructions or accessing information that may be stored in memory. The functionalities of multiple servers may be integrated into a single server. Any of the aforementioned servers (or an integrated server) may take on certain client-side, cache, or proxy server characteristics. These characteristics may depend on the particular network placement of the server or certain configurations of the server.

Collectively, cloud hub/storage device 126, pathogen detection system 140 (including application 142), and database resources 132 (including various database 134A-134N) represent remote and/or distributed computing resources that may be used to implement any of the steps of the methods described herein. Processing resources, for example, associated with cloud hub/storage device 126, pathogen detection system 140 (including application 142), and database resources 132 (including various database 134A-134N) may be used to perform the various analyses, filters, comparisons, matches, etc., that may be applied to the sample sequence as sequenced by sequencing system 102. Further, cloud hub/storage device 126, pathogen detection system 140 (including application 142), and database resources 132 (including various database 134A-134N) may be used to store the sequencing data (including backup copies thereof) provided by sequencing system 102. Such sequencing data may include the full sequence, partial sequences (e.g., subsets or portions of sequences), data regarding sequence matches, and combinations thereof.

Additionally, cloud hub/storage device 126, pathogen detection system 140 (including application 142), and database resources 132 (including various database 134A-134N) may provide sequence libraries that may be used to filter and compare the sample sequence as sequenced by sequencing system 102. Such libraries may be updated over time as new genetic sequences and variants arise, thus allowing for comparison to an ever-increasing body of genetic sequences. Such libraries may be used to filter the sample sequence as sequenced by sequencing system 102 to exclude sequences that are not of interest (e.g., contaminants or known non-pathogenic sequences), as well as identify matches to sequences that are of interest (e.g., sequences with percent identity to conserved pathogenic sequences). Such designations of interest may be made by administrators, manufacturers, operators, users, and other individuals associated with use and maintenance of sequencing systems. Conserved pathogenic sequences that may be compared to identify sequences of interest include but are not limited to viral replication genes, conserved regions of viral hemagglutinin genes, viral nucleoprotein genes, viral matrix protein genes, viral or bacterial polymerase genes, bacterial ribosome genes, bacterial tmRNA, bacterial heat shock protein genes, bacterial metabolic genes, and bacterial cytoskeleton genes. For example, conserved pathogenic sequences may include SARS-COV2 REP, SARS-COV REP, influenza PB1, influenza PB2, influenza PA, influenza NP, influenza M1, HIV capsid, HIV nucleocapsid, HIV protease, HIV RT, HIV integrase, HIV VRP, HIV GP41, Bacillus anthracis pXO1, or fragments thereof.

Where the genetic sequence of interest pertains to pathogens, excluding certain sequences (e.g., human DNA, non-human animal DNA, plant DNA, other host DNA) may limit the size of the genetic sequence to be analyzed from the sample. In some embodiments, therefore, a genetic sequence of the sample as sequenced by sequencing system 102 may be filtered to exclude portions matching known sequences from specified categories that are not of interest. Filtering may be performed by a filtering module, such as filtering module 144 of pathogen detection system 140. While sequencing system 102 and pathogen detection system 140 are illustrated as separate systems, one skilled in the art would understand that sequencing system 102 and pathogen detection system 140 may be executing on the same side of the server. Only the filtered subset(s) remaining may thereafter be subject to comparisons against known pathogenic sequences. By limiting the amount of genetic sequence being processed, the efficiency and speed of processing the remaining subset(s) may be improved, as well as requiring less room for storage of the same. Conversely, libraries may also be used to reduce the genetic sequence to focus on sequences that are of interest. For example, libraries that include genetic sequences related to Potential Pandemic Pathogens (PPP) may be used to identify any matches to known pathogenic sequences.

The cloud hub/storage device 126, pathogen detection system 140 (including application 142), and database resources 132 (including various database 134A-134N) may further store libraries to be used to identify whether the sample sequence matches any sequences of interest (e.g., pathogenic genetic sequences). To determine whether a sequence (e.g., a sequence read from a sequencer) corresponds to a pathogenic sequence, the sequence can be compared to one or more libraries of known pathogenic sequences, which may include various public, private, and commercial databases, using a comparison module (e.g., comparison module 146 of pathogen detection system 140) to evaluate the similarity between the sequence and a known pathogenic sequence from the library. While sequencing system 102 and pathogen detection system 140 are illustrated as separate systems, one skilled in the art would understand that sequencing system 102 and pathogen detection system 140 may be executing on the same side of the server. Such libraries and databases may include the Potential Pandemic Pathogens (PPP) database, Virus Pathogen Resource (ViPR), NCBI BLAST, NCBI Virus, Oxford University viruSITE, NCBI Viral Genomes Resource, INSDC database, NIH/NCBI GenBank, European Nucleotide Archive (ENA), protein families (Pfam) database, Conserved Domain Database, and DNA Databank of Japan (DDBJ).

The comparison threshold may be expressed as a minimum percent identity (% ID) over a minimum sequence length. The threshold % ID relative to the pathogenic sequence needed to still qualify as the pathogen may vary, because as noted in “viral sequencing” above, certain proteins (and therefore the associated nucleic acid regions within a viral genome) are more highly conserved than others within a viral genus (e.g., the betacoronavirus genus containing SARS-COV-2, MERS-COV, and SARS-COV). For Influenza A, for example, the most highly conserved sequences correspond to non-structural proteins (e.g., transcription machinery proteins like PB1, PB2, and PA). Meanwhile, surface proteins (e.g., HA and NA of influenza) may be less conserved, since varying surface proteins may help the pathogen evade the host immune response. The threshold % ID may vary based on the pathogen of interest being examined, and thus, different threshold % ID values may be used to identify what is considered to be matching sequence as discussed herein. For example, a threshold % ID may be set at a minimum of 80% identity. In another example, a minimum threshold may be set based on a viral recombination limit for viral sub-genera, such as a minimum of about 90% sequence identity. The threshold % ID may be based on an expected level of variation that may occur within a pathogen (e.g., a viral sub-genera) while maintaining the ability to recombine with another member of the same species or sub-genera. The threshold minimum % ID may be determined over a minimum length, such as the length of a single sequencing read.

In some embodiments, a threshold % ID may be set at no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 97.50%, no less than about 98%, no less than about 98.50%, no less than about 99%, no less than about 99.50%, no less than about 99.70%, no less than about 99%, or no less than about 100%. In some embodiments, a minimum length over which % ID may be determined may be no less than about 10, no less than about 15, no less than about 20, no less than about 25, no less than about 30, no less than about 35, no less than about 40, no less than about 45, no less than about 50, no less than about 55, no less than about 60, no less than about 65, no less than about 70, no less than about 75, no less than about 80, no less than about 85, no less than about 90, no less than about 95, no less than about 100, no less than about 120, no less than about 140, no less than about 160, no less than about 180, no less than about 200, no less than about 220, no less than about 240, no less than about 260, no less than about 280, no less than about 300, no less than about 350, no less than about 400, no less than about 450, or no less than about 500 nucleotide bases. In some embodiments, a minimum length over which % ID may be determined may be no less than about 5, no less than about 10, no less than about 12, no less than about 15, no less than about 17, no less than about 20, no less than about 22, no less than about 25, no less than about 30, no less than about 35, no less than about 40, no less than about 45, no less than about 50, no less than about 60, no less than about 70, no less than about 80, no less than about 90, no less than about 100, no less than about 120, no less than about 140, no less than about 160, no less than about 180, or no less than about 200 amino acid residues.

While the foregoing discussion generally pertains to a genetic sequence of nucleic acids, similar analyses may be performed on a corresponding peptide sequence associated with the nucleic acid sequence. For example, the nucleic acid sequence may be converted to a peptide sequence (3 different peptide sequences corresponding to 3 possible reading frames) and compared to a protein sequence database. As viruses often use frame shifts to code multiple proteins with a single nucleic acid sequence, such a conversion may likewise incorporate such frame shifts into different peptide sequences for further analyses and comparisons. Percent sequence identity of a nucleic acid or peptide sequence can be determined by conventional methods. See, for example, Altschul et al., Bull. Math. Bio. 48:603 (1986), and Henikoff and Henikoff, Proc. Natl. Acad. Sci. USA 89:10915 (1992). In some embodiments, the sequence identity may be calculated as: ([Total number of identical matches]/[length of the longer sequence plus the number of gaps introduced into the longer sequence in order to align the two sequences])(100). Various methods and software programs can be used to determine the homology between two or more nucleic acids or peptides, such as NCBI BLAST, Clustal W, MAFFT, Clustal Omega, AlignMe, Praline, or another suitable method or algorithm.

Additionally, there are many established algorithms available to align two nucleic acid sequences. For example, the “FASTA” similarity search algorithm of Pearson and Lipman is a suitable nucleic acid alignment method for examining the level of sequence identity shared by a nucleic acid sequence. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., a nucleotide sequence received from a sequencing read) and a test sequence (e.g., a reference pathogenic sequence) that has either the highest density of identities or pairs of identities. For nucleotide sequence comparisons, the ktup value can range between one to six, preferably from three to six, most preferably three. The ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. These parameters can be introduced into a FASTA program by modifying the scoring matrix file (“SMATRIX”), as explained in Appendix 2 of Pearson, Meth. Enzymol. 183:63 (1990). FASTA can also be used to determine the sequence identity or homology of amino acid molecules using a ratio as disclosed above. For amino acid sequences, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62.

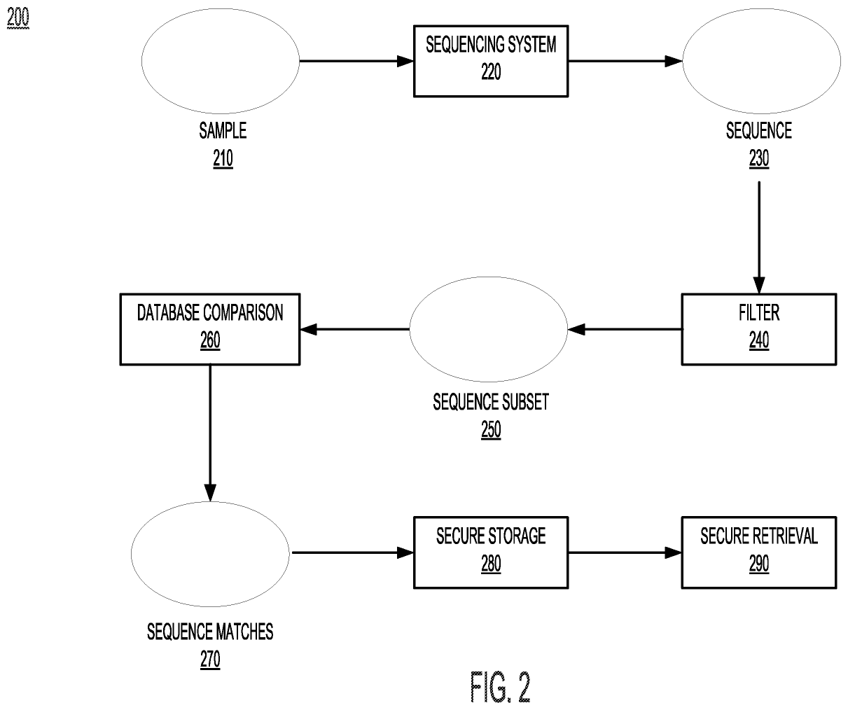

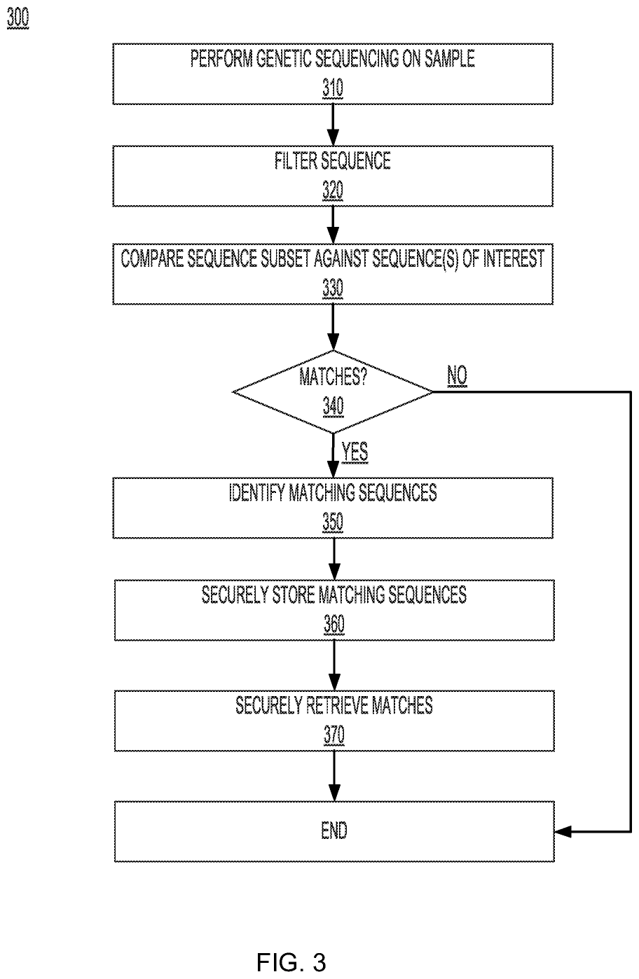

FIG. 2 is a data flow diagram illustrating an exemplary flow of data 200 in a method for pathogenic sequence identification 300, which is illustrated in the flowchart of FIG. 3. In exemplary embodiments, an initial sample 210 of genetic material may be sequenced, filtered, analyzed, matched to identified sequences, and secured during performance of the method 300. As discussed herein, the flow of data 200 may occur locally within the sequencing system 102 (and associated peripheral and other local devices) or may use remote computing resources available over a communication network.

As illustrated, an initial sample 210 may be prepared and provided to sequencing system 220 for sequencing in step 310 of the method 300. Sequencing system 220 (which may correspond to the sequencing system 102 as illustrated and described in relation to FIG. 1) may therefore perform various sequencing techniques as discussed herein and produce one or more files with a full genetic sequence dataset 230. As discussed above, the genetic sequence 230 may include not only the genetic sequence of interest, but genetic sequences related to contaminants or other sources that are not of interest.

In step 320 of the method 300 of FIG. 3, the full genetic sequence dataset 230 may be subject to a filter 240. Such a filter 240 may include a filtering algorithm that compares the full genetic sequence 230 to one or more libraries of sequences determined not to be of interest. Filtering may be performed by a filtering module, such as filtering module 144 of pathogen detection system 140. While sequencing system 102 and pathogen detection system 140 are illustrated as separate systems, one skilled in the art would understand that sequencing system 102 and pathogen detection system 140 may be executing on the same side of the server. Where the interest is in pathogens, therefore, the libraries may include non-pathogenic sequences. Thus, such sequences-that may be associated with contamination in the pathogenic sequence sample-may be identified and excluded from further analysis. Such exclusion may include discarding such sequences, which reduces the full genetic sequence 230 into a filtered sequence subset 250. Such reduction may reduce processing load and storage requirements by limiting the size of genetic sequences that are subject to further analysis and storage.

In step 330, the filtered sequence subset 250 may be compared to specified libraries of sequences determined to be of interest. The database comparison 260 may be performed by a filtering module, such as filtering module 144 of pathogen detection system 140. While sequencing system 102 and pathogen detection system 140 are illustrated as separate systems, one skilled in the art would understand that sequencing system 102 and pathogen detection system 140 may be executing on the same side of the server. Where the interest is in identifying pathogenic sequences, the libraries may include known genetic sequences related to Potential Pandemic Pathogens (PPP). Such comparison may yield a determination of whether the filtered sequence subset 250 includes sequences matching that of known pathogens in step 340. As discussed above, different comparison thresholds may be applied to identify what is considered a match. A specified threshold % ID may be used to identify which portions of the filtered sequence subset 250 are considered matches and which portions are not considered matches.

Where no matches are identified in step 340, the method may end without further analysis. In such instances, the data regarding the initial sample 210 may be discarded so as to preserve processing and storage resources for actual matches. Where such resources are distributed over a network, the data reduction may further allow for more efficient bandwidth usage. Where one or more matches are identified in step 340, however, the method may proceed to step 350.

In step 350, portions of the filtered sequence subset 250—the sequence matches 270—have been identified as matching known sequence portions (e.g., pathogenic sequences). Thus, the initial sample 210 may be determined to include sequence matches 270 to genetic sequences of known pathogens. In some implementations, sequences within the filtered sequence subset 250 that are not matches may be discarded, which may preserve processing, storage, and bandwidth resources for use in relation to the sequence matches 270.

In step 360, the sequence matches 270 may be securely stored in a secure location, such as secure storage 280, which may be inclusive of any of the databases and storage devices discussed herein. In some embodiments, the sequence matches 270 may be subject to further data compression before being stored in accordance with compression techniques known in the art. Further, while a sequence match 270 may have been determined to match a known pathogenic genetic sequence, a certain level of differences (e.g., in nucleotide identity) may exist. Rather than storing the entire sequence match 270 therefore, an identifier of the associated pathogen or matching pathogenic sequence may be stored, along with an identifier of the differences. The data may also be stored in password-protected and/or encrypted form and under rules and policies that limit access rights to designated authorized individuals. As such, only the designated authorized individuals may be permitted to retrieve and/or decrypt the match data, such as via secure retrieval 290, in step 370.

The method 300 of FIG. 3 may be embodied as executable instructions in a non-transitory computer readable storage medium including but not limited to a CD, DVD, or non-volatile memory such as a hard drive. The instructions of the storage medium may be executed by a processor (or processors) to cause various hardware components of a computing device hosting or otherwise accessing the storage medium to effectuate the method. The steps identified in FIG. 3 (and the order thereof) are exemplary and may include various alternatives, equivalents, or derivations thereof including but not limited to the order of execution of the same. Thus, while processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times. Further any specific numbers noted herein are only examples: alternative implementations may employ differing values or ranges.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### EXAMPLES

The invention is further illustrated by the following non-limiting examples.

### Example 1

### Identification of a Potential Pandemic Pathogen Sequence

This example describes identification of a potential pandemic pathogen (PPP) sequence from a sequencing dataset. A sample containing DNA is sequenced (e.g., using high throughput next generation sequencing), and a sequence file is generated that contains genetic sequence data. The sequence file is stored on a storage device connected to or networked with the sequencing instrument. Upon storage of the sequence file to the storage device, software associated with the sequencing device automatically compares genetic sequence data in the sequence file to a library of reference sequences containing reference sequences, including human genomic sequences, that are not of interest. Sequences from the sequence file that do not match the references sequences with at least about 90% sequence identity over a length of at least about 100 nucleotides are designated as a filtered sequence subset. The filtered sequence subset is then compared to a library of highly conserved pathogenic sequences of interest, including sequences that encode highly conserved viral proteins of pathogenic viruses such as SARS-COV-2.

Sequences that match or contain a region that match a conserved pathogenic sequence with at least about 90% sequence identity over a length of at least about 100 nucleotides are identified, and data regarding the identified sequence and/or sequence identity relative to the pathogenic sequence is securely stored locally or in a secure database. The stored data may be accessed at a later time by researchers or public health officials studying pandemic origins.

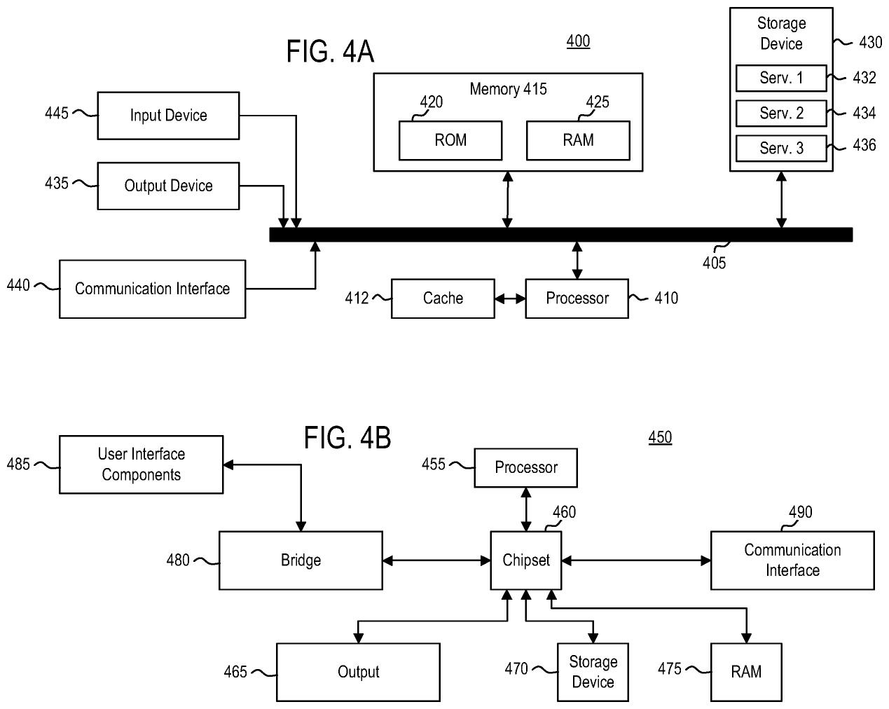

FIG. 4A illustrates a system bus architecture of computing system 400, according to example embodiments. System 400 may be representative of at least a portion of pathogen detection system 140. One or more components of system 400 may be in electrical communication with each other using a bus 405. System 400 may include a processing unit (CPU or processor) 410 and a system bus 405 that couples various system components including the system memory 415, such as read only memory (ROM) 420 and random access memory (RAM) 425, to processor 410. System 400 may include a cache of high-speed memory connected directly with, in close proximity to, or integrated as part of processor 410. System 400 may copy data from memory 415 and/or storage device 430 to cache 412 for quick access by processor 410. In this way, cache 412 may provide a performance boost that avoids processor 410 delays while waiting for data. These and other modules may control or be configured to control processor 410 to perform various actions. Other system memory 415 may be available for use as well. Memory 415 may include multiple different types of memory with different performance characteristics. Processor 410 may include any general purpose processor and a hardware module or software module, such as service 1 432, service 2 434, and service 3 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction with the computing system 400, an input device 445 may represent any number of input mechanisms, such as a microphone for speech, a touch-sensitive screen for gesture or graphical input, keyboard, mouse, motion input, speech and so forth. An output device 435 may also be one or more of a number of output mechanisms known to those of skill in the art. In some instances, multimodal systems may enable a user to provide multiple types of input to communicate with computing system 400. Communications interface 440 may generally govern and manage the user input and system output. There is no restriction on operating on any particular hardware arrangement and therefore the basic features here may easily be substituted for improved hardware or firmware arrangements as they are developed.

Storage device 430 may be a non-volatile memory and may be a hard disk or other types of computer readable media which may store data that are accessible by a computer, such as magnetic cassettes, flash memory cards, solid state memory devices, digital versatile disks, cartridges, random access memories (RAMs) 425, read only memory (ROM) 420, and hybrids thereof.

Storage device 430 may include services 432, 434, and 436 for controlling the processor 410. Other hardware or software modules are contemplated. Storage device 430 may be connected to system bus 405. In one aspect, a hardware module that performs a particular function may include the software component stored in a computer-readable medium in connection with the necessary hardware components, such as processor 410, bus 405, output device 435 (e.g., display), and so forth, to carry out the function.

FIG. 4B illustrates a computer system 450 having a chipset architecture that may represent at least a portion of pathogen detection system 140. Computer system 450 may be an example of computer hardware, software, and firmware that may be used to implement the disclosed technology. System 450 may include a processor 455, representative of any number of physically and/or logically distinct resources capable of executing software, firmware, and hardware configured to perform identified computations. Processor 455 may communicate with a chipset 460 that may control input to and output from processor 455. In this example, chipset 460 outputs information to output 465, such as a display, and may read and write information to storage device 470, which may include magnetic media, and solid state media, for example. Chipset 460 may also read data from and write data to storage device 475 (e.g., RAM). A bridge 480 for interfacing with a variety of user interface components 485 may be provided for interfacing with chipset 460. Such user interface components 485 may include a keyboard, a microphone, touch detection and processing circuitry, a pointing device, such as a mouse, and so on. In general, inputs to system 450 may come from any of a variety of sources, machine generated and/or human generated.

Chipset 460 may also interface with one or more communication interfaces 490 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 455 analyzing data stored in storage device 470 or storage device 475. Further, the machine may receive inputs from a user through user interface components 485 and execute appropriate functions, such as browsing functions by interpreting these inputs using processor 455.

It may be appreciated that example systems 400 and 450 may have more than one processor 410 or be part of a group or cluster of computing devices networked together to provide greater processing capability.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for pathogenic sequence identification, the method comprising:
receiving a genetic sequence file from a sequencing system, the genetic sequence file including a genetic sequence associated with a sample;
filtering the genetic sequence associated with the sample to exclude at least one sequence portion determined not to be of interest, wherein filtering the genetic sequence results in a filtered sequence subset;
identifying that at least one portion of the filtered sequence subset includes one or more matches to sequences of interest; and
storing data regarding the identified matches in memory.

2. The method of claim 1, wherein filtering the genetic sequence includes comparing the genetic sequence to a library of sequences designated as not being of interest, and wherein the at least one excluded sequence portion matches one of the sequences in the library.

3. The method of claim 1 or claim 2, wherein identifying the matches includes comparing the filtered sequence subset to a library of sequences designated as being of interest, and wherein the identified matches correspond to the sequences in the library.

4. The method of any one of claims 1-3, further comprising encrypting the data regarding the identified matches, wherein the stored data is encrypted.

5. The method of claim 4, further comprising retrieving the stored data in response to a request by a requestor, and wherein retrieving the stored data includes decrypting the encrypted data.

6. The method of any one of claims 1-5, further comprising retrieving the stored data includes verifying that a requestor is authorized to access the stored data.

7. The method of any one of claims 1-6, further comprising identifying that the matches are associated with a known pathogen.

8. The method of any one of claims 1-7, wherein storing the data regarding the identified matches includes storing an identifier of the known pathogen.

9. The method of any one of claims 1-8, wherein storing the data regarding the identified matches includes storing an identifier of one or more sequences of the known pathogen that correspond to the matches.

10. The method of any one of claims 1-9, wherein storing the data regarding the identified matches includes storing an identifier of one or more sequences of the known pathogen that do not correspond to the matches.

11. A system for pathogenic sequence identification, the system comprising:
a processor; and
a memory having programming instructions stored thereon, which, when executed by the processor causes the system to perform operations comprising:
receiving a genetic sequence file from a sequencing system, the genetic sequence file including a genetic sequence associated with a sample,
filtering the genetic sequence associated with the sample to exclude at least one sequence portion determined not to be of interest, wherein filtering the genetic sequence results in a filtered sequence subset, and
identifying that at least one portion of the filtered sequence subset includes one or more matches to sequences of interest.

12. The system of claim 11, wherein the processor filters the genetic sequence by comparing the genetic sequence to a library of sequences designated as not being of interest, and wherein the at least one excluded sequence portion matches one of the sequences in the library.

13. The system of claim 11 or claim 12, wherein the processor identifies the matches by comparing the filtered sequence subset to a library of sequences designated as being of interest, and wherein the identified matches correspond to the sequences in the library.

14. The system of any one of claims 11-13, wherein the processor executes further instructions to encrypt the data regarding the identified matches, wherein the stored data is encrypted.

15. The system of claim 14, wherein the processor executes further instructions to retrieve the stored data in response to a request by a requestor and to decrypt the encrypted data.

16. The system of any one of claims 11-15, wherein the processor executes further instructions to retrieve the stored data by verifying that a requestor is authorized to access the stored data.

17. The system of any one of claims 11-16, wherein the processor executes further instructions to identify that the matches are associated with a known pathogen.

18. The system of any one of claims 11-17, wherein the memory stores the data regarding the identified matches by storing an identifier of the known pathogen.

19. The system of any one of claims 11-18, wherein the memory stores the data regarding the identified matches by storing an identifier of one or more sequences of the known pathogen that correspond to the matches.

20. The system of any one of claims 11-19, wherein the memory stores the data regarding the identified matches by storing an identifier of one or more sequences of the known pathogen that do not correspond to the matches.

21. A non-transitory, computer-readable storage medium, having embodied thereon a program executable by a processor to perform a method for pathogenic sequence identification, the method comprising:
receiving a genetic sequence file from a sequencing system, the genetic sequence file including a genetic sequence associated with a sample;
filtering the genetic sequence associated with the sample to exclude at least one sequence portion determined not to be of interest, wherein filtering the genetic sequence results in a filtered sequence subset;
identifying that at least one portion of the filtered sequence subset includes one or more matches to sequences of interest; and
storing data regarding the identified matches in memory.
